(19) United States
(12) Patent Application Publication
(10) Pub. No.: US 2010/0004193 A1
(43) Pub. Date: Jan. 7, 2010

(54) COMBINATION THERAPY

(75) Inventors: Reimar ABRAHAM, Padova (IT); Stefano SCHIAFFINO, Padova (IT); Karla HAJMAN, Stockholm (SE); Emanuele MINATTI, Saonara (IT)

Correspondence Address: YOUNG & THOMPSON, 209 Madison Street, Suite 500, Alexandria, VA, 22314, US

(73) Assignees: FONDAZIONE PER LA RICERCA BIOMEDICA AVANZATA ONLUS - ISTITUTO VENETO DI MEDICINA MOLECOLARE, Padova (IT); UNIVERSITA' DEGLI STUDI DI PADOVA, Padova (IT)

(21) Appl. No.: 12/497,882

(22) Filed: Jul. 6, 2009

Related U.S. Application Data
(60) Provisional application No. 61/078,470, filed on Jul. 7, 2008.

(51) Int. Cl. A61K 31/7068; A61K 31/715

(52) U.S. Cl. 514/49; 514/54

(57) ABSTRACT

A method of treating a subject affected by neoplastic diseases includes the administration to the subject of an aminoglycoside antibiotic in combination with a nucleoside analogue.

The present invention concerns a method of treatment of a subject affected by neoplastic diseases comprising the administration to said subject of an aminoglycoside antibiotic in combination with nucleoside analogs. Geneticin (G418 sulfate, G418) is a preferred aminoglycoside antibiotic and Gemcitabine or Cytarabine are preferred nucleoside analogs.

The invention also concerns pharmaceutical compositions comprising gemcitabine and geneticine as the active ingredients.

Abstract

Although many cancer drugs damage DNA, the mechanisms leading to DNA damage-induced cell death in tumour cells are poorly defined. In order to identify new elements of DNA damage-induced cell death, we generated 23 clonal bladder cancer cell lines that varied in sensitivity to the DNA-damaging drug Gemcitabine. Gene expression profiling revealed elevated expression of protein synthesis-controlling genes in all Gemcitabine-resistant clones thus suggesting that increased protein translation underlies resistance. Metabolic labelling studies showed that protein synthesis in the presence of Gemcitabine was indeed increased in the resistant clones. Overexpression of the translation factor EIF4E3 increased global protein synthesis and inhibited cell death by Gemcitabine and the DNA-damaging drugs Mitomycin C and etoposide but not cisplatin. Accordingly, concomitant treatment with the protein synthesis inhibitor G418 synergistically decreased protein synthesis rates, reversed Gemcitabine-resistance and prevented the selection of resistant clones. Furthermore, in cancer cell lines of diverse origin, addition of G418 lowered the EC-50 value for cell death by Gemcitabine up to 30 fold. Our results identify regulation of overall protein synthesis as a vital component of DNA damage signaling and suggest a rational drug combination that enhances Gemcitabine action.

### BACKGROUND OF THE INVENTION

The repair of DNA is a fundamental requirement for the correct transmission of the genetic information and hence for the survival of all organisms. How DNA damage is recognized and repaired is an area of intense research and many details of this process have been elucidated (Kastan & Bartek, 2004).

In contrast, relatively little is known about the signals that initiate cell death upon DNA damage. Cells die after a gross DNA damage that cannot be sufficiently repaired and cell death is therefore a fail-safe mechanism that prevents the accumulation of DNA damage by eliminating the damaged cell. The apoptotic response after DNA damage depends on the members of the p53 family of proteins, p53, p63 and p73. It has been shown that in normal cells, p53 is required for cell death induced by DNA damaging agents. Upon damage, p53 is stabilized leading to p53-mediated transcription of pro-apoptotic genes like the Bcl-2 homologs PUMA and NOXA or genes that code for members of the death-receptor pathway like Fas/CD95 or death receptor 5 (Harris & Levine, 2005; Vousden, 2000).

Since the p53 pathway is deactivated in many if not all cancers (Vogelstein & Kinzler, 2004), alternative ways must exist that lead to cell death upon DNA damaging drugs in cancer cells. It is generally thought that an alternative route of cell death induction proceeds via the stress kinase JNK and the tyrosine kinase c-abl (Davis, 2000; Kharbanda et al., 1997; Yuan et al., 1997). Recently, p53-independent cell death was also shown via DNA damage-induced deactivation of cyclin-dependent-kinase 2 (CDK2) preventing phosphorylation and inhibition of the pro-apoptotic FOXO transcription factors by this kinase (Huang et al., 2006).

In recent years, the notion that aberrant protein synthesis regulation plays a role in cancer formation has gained strong support. Many ribosomal proteins as well as translation initiation and elongation factors are overexpressed in various cancer types. Prominently investigated is the eukaryotic translation initiation factor 4E (EIF4E) whose forced overexpression in mouse fibroblasts transforms the cells and whose constitutive overexpression in mice leads to enhanced tumourigenesis (Ruggero & Pandolfi, 2003). EIF4E is activated by the Akt/mTOR pathway that comprises the proto-oncogene Akt and the negative regulators and tumour suppressors PTEN and TSC1/2 (Hay & Sonenberg, 2004) linking protein translation to genetic defects in cancer. Increased protein synthesis may lead to the preferential translation of specific proteins that contribute to cancer formation like c-myc, cyclin D1 or fibroblast growth factor (De Benedetti & Graff, 2004; Stoneley & Willis, 2004).

DNA damage has been shown to influence overall protein synthesis. Treatment of mouse fibroblasts with DNA-damaging drugs can lead to a decrease in protein synthesis but its function in the DNA damage response remains elusive (Tee & Proud, 2000).

Gemcitabine (2′-2′-difluorodeoxycytidine) is a nucleoside analog that is used for the treatment of non-small cell lung cancer, pancreatic cancer, bladder cancer, breast cancer and ovarian cancer. Gemcitabine (Gem) is hydrophilic and therefore not a substrate of the multi-drug resistance transporter proteins P-glycoprotein and MDR which export hydrophobic drugs out of the cell. Instead, Gem is transported across the plasma membrane by nucleoside transporters. Inside the cell, Gem is phosphorylated by nucleoside kinases to yield the active diphosphate and triphosphate nucleosides which inhibit ribonucleotide reductase (diphosphate) and incorporate into DNA (triphosphate) during S-phase of the cell cycle leading to inhibition of DNA polymerization, S-phase arrest and subsequent cell death (Barton-Burke, 1999; Plunkett et al., 1996). Resistance to Gem under experimental settings has been attributed to decreased expression of the Gem-phosphorylating kinases or to stronger expression of ribonucleotide reductase (Bergman et al., 2005; Bergman et al., 2002).

We set out to identify genetic modulators of Gem-induced cell death that may influence the DNA damage response in general. To this end, we employed a functional genomics strategy that compares, by means of DNA microarrays, clonal cell lines that differ in their sensitivity towards Gem (Abraham et al., 2005; Kang et al., 2003). We chose bladder cancer cell lines for our scope because bladder cancer is one of the cancer types where Gemcitabine (in combination with cisplatin) is used as a therapy of metastatic cancer. Taken together, our results implicate protein synthesis regulation as an essential component of the DNA damage response and suggest a novel, rational cancer combination therapy.

### DESCRIPTION OF THE INVENTION

It has now been surprisingly found that combination of nucleotide analogs such as gemcitabine, Ara-C and similar compounds with aminoglycoside antibiotics such as geneticine, gentamycine, kanamycine, streptomycin and the like can impair the survival of clones of tumor cells resistant to gemcitabine or other nucleotide analogs, particularly in non-small cell lung cancer, pancreatic cancer, bladder cancer, breast cancer and ovarian cancer cells.

The present invention accordingly provides a method of treatment of a subject affected by neoplastic diseases comprising the administration to said subject of an aminoglycoside antibiotic, preferably geneticine, in combination with a nucleotide analogue, preferably gemcitabine or Ara-C.

The invention also provides pharmaceutical compositions comprising gemcitabine or Ara-C and geneticine as the active ingredients.

### DETAILED DESCRIPTION OF THE INVENTION

### Materials and Methods

Reagents—Gem was purchased from Eli Lilly, Mitomycin C from Kyowa Italiana Farmaceutici, etoposide and cisplatin from Sigma. The Bcl-2 antibody was from Santa Cruz, the β-Actin antibody from Sigma. All secondary antibodies were from Biorad. The Bcl-2 expression plasmid was a gift from L. Scorrano, Venetian Institute of Molecular Medicine. The caspase inhibitor z-VAD-fmk (ZVAD) was from Alexis, G418 from Invitrogen and cycloheximide from Sigma. Rapamycin was a kind gift from Wyeth Pharmaceuticals. [³⁵S]-Methionine/Cysteine was acquired as Revidue Pro-Mix from GE Healthcare/Amersham.

Cell culture, cell death assays and growth assays—The T-24 and HT-1376 cell lines were purchased from the German Collection of Microorganisms and Cell Cultures and were cultivated in DMEM, high glucose, containing 10% Fetal Bovine Serum (FBS) or Minimum Essential Medium with 10% FBS and non-essential amino acids, respectively. HT-1197 was a gift from Bioxell (Milano) and was cultured like HT-1376. T-24 and its clones were incubated for 2 days with Gem, etoposide, Mitomycin C or cisplatin. HT-1376 and its clones were incubated for 4 days and HT-1197 for 5 days with Gem including one medium change after 2 days. The percentage of dead cells that took up propidium iodide was determined using flow cytometry. Background values of control cells (no treatment for Gem, Mitomycin C, G418, cycloheximide, cisplatin; DMSO for ZVAD, etoposide) were subtracted. Staining with Annexin-V-FITC was performed according to the manufacturer's description (Roche). To assess the cell number by staining with 3-(4,5-Dimethylthiazol-2-yl)-2,5-diphenyltetrazolium bromide (MTT), MTT was added at 500 μg/ml. After 2 hours, precipitate was solubilized by addition of 0.04N HCl/Isopropanol. Absorbance was read at 570 nm with reference at 650 nm.

Generation of Gemcitabine-resistant clones and stable cell lines—The T-24 cell line was incubated with 120 nM Gem for 3 days, HT-1376 with 160 nM Gem and HT-1197 with 1.6 μM for 7 days. We chose these concentrations because higher concentrations did not yield clones while lower concentrations yielded too many non-separated colonies. Subsequently, the medium was changed for normal growth medium without Gem and the cells were grown for additional 1 to 3 weeks. Cell clones were picked and tested for Gem sensitivity and cell growth rates by seeding the clones at the same cell number and performing cell death assays as described above. As a control, the cell lines were seeded at low concentrations that allowed the growth of cell clones without Gem treatment. Gem-resistance was defined by a reduction in cell death of at least 50% compared to the parental cells. We used non-selected sensitive clones as well as Gem selected, but sensitive clones as the control group for the Gem selected resistant clones. EIF4E3 was cloned by PCR using primers flanking the coding sequence and cDNA from a resistant clone of HT-1197 as a template. The construct was sequenced and found to be identical with the published DNA sequence. T-24 cells were transfected with the empty expression plasmid (pcDNA3.1) or the construct containing EIF4E3 and selected using G418.

DNA microarray hybridization and statistical analysis—RNA was isolated from clones cultured without Gem using an RNeasy kit (Qiagen). Subsequent steps were carried out according to the manufacturers protocols (Affymetrix). U133 Plus 2.0 GeneChip arrays were used which interrogate the expression of 47000 transcripts. Raw data were converted using Mas 5.0. For finding groups of genes that were differentially expressed, we used the Global test by Goeman et al. (Goeman et al., 2004). The p-values were adjusted for multiple hypothesis testing as described (Benjamini & Hochberg, 1995). For determining differentially expressed genes, the Pearson correlation with the cell death profile of the respective clone set was calculated. This way, changes in cell death level within the groups of sensitive or resistant clones were also taken into account. High correlation factors mean that the expression of the respective gene is high when the cell death level is high. Low correlation factors mean that the expression of the respective gene is high when the cell death level is low. We set the threshold for genes that are upregulated in the resistant clones at r=−0.8 or lower and the threshold for genes that are downregulated in the resistant clones at r=0.8 or higher. We found 122 genes upregulated in the resistant clones of the T-24 clone set, 347 genes in the HT-1197 clone set and 354 genes in the HT-1376 clone set. For gene group enrichment analysis we used the Gene Ontology tool at the Affymetrix/Netaffx website. Except for the dose response curves, all P-values were determined using student's t-test. All statistical analysis and curve fitting was performed using GraphPad Prism.

Metabolic labelling—Labelling was performed after one day Gem, Mitomycin C, etoposide or cisplatin for the T-24 cell line and its clones and 3 days Gem for the HT-1376 and HT-1197 cell lines. Cells were labelled for 1.5 hours in growth medium without methionine and cysteine supplemented with 30 μCi/ml [³⁵S]-methionine/cysteine. Subsequently, the proteins were precipitated with TCA as described previously (Zhang et al., 2004). The incorporated radioactivity was expressed as counts/μg protein.

Quantitative real-time PCR and immunoblotting—We used the same RNA that was isolated for the DNA microarrays and prepared cDNA using Applied Biosystem (ABI) Reverse Transcription Reagents with random hexamer primers. Real-time PCR was performed using the PCR Master Mix (ABI) and ready-made TaqMan expression assays on an ABI Prism 7000. We used GAPDH expression for normalizing the expression values except for the T-24 clone set where GAPDH was slightly upregulated in the resistant clones (as measured by DNA microarray and repeated quantitative real-time PCR). For this clone set, we used another “house-keeping gene”, HPRT1, for normalizing the expression values. Expression levels were calculated according to the formula: 1000*2^{(Ct target gene-Ct house keeping)}. Cell lysis for protein extraction was performed on the same day as metabolic labelling. Immunoblotting was done as described previously (Abraham et al., 2005).

Cell cycle analysis—Cell cycle analysis was performed by flow cytometry as described before (Nicoletti et al., 1991) on the same day as metabolic labelling. The cell cycle distribution was determined using the ModFit programme.

Results

Selection of Gem resistant clones—We chose three different bladder cancer cell lines for our scope that were isolated from high grade, transitional bladder cancer: T-24, HT-1197 and HT-1376 (Bubenik et al., 1973; Rasheed et al., 1977). In order to elucidate novel steps in Gem-induced cell death, we set out to derive cell clones from these cell lines that differed in their sensitivity towards Gem and whose gene expression profiles could then be correlated with Gem sensitivity. For selecting Gem-resistant clones, we incubated the three cell lines with Gem for 3 to 7 days. After this time, the drug was removed but cells kept on dying. Around 50 clones were recovered per 1 million seeded cells. As control sensitive clones, we derived clonal cell lines from each parental cell line by limiting dilution without Gem selection.

We tested about 100 clones for their sensitivity towards Gem in shorter assays of 2 to 5 days Gem exposure. This time depended on the growth rate of cells. T-24 that had the highest growth rate also died earlier. For further studies, we chose three sets of clones consisting of 4 sensitive and 4 resistant clones (T-24 and HT-1197 clone sets) or 3 sensitive and 5 resistant clones (HT-1376 clone set) derived from each parental cell line. The clones showed various degrees of sensitivity towards Gem induced cell death; some clones were completely resistant under our assay conditions. Importantly, the groups of sensitive and resistant clones of each clone set did not differ in their proliferation rates (FIG. 1). Hence, the reduced cell death measured at one time point was not merely due to decreased Gem incorporation because fewer cells had passed S-phase.

Gem induced an apoptotic form of cell death that proceeded via engagement of mitochondria in T-24 and HT-1197 as Bcl-2 overexpression could inhibit cell death. In HT-1376, apoptosis could not be inhibited by Bcl-2. However, this might be attributed to the relatively low overexpression of Bcl-2 (Supplementary FIG. 1).

Protein synthesis controlling genes are upregulated in the resistant clones—To investigate the genetic basis of the Gem-resistant phenotype, we measured the gene expression without Gem treatment using whole genome DNA microarrays. As a first step, we identified for each clone set the single genes that were differentially expressed in the resistant and sensitive clones and hence potentially contributed to resistance formation. To verify that our strategy worked in identifying genes that influence drug resistance, we performed a literature search of 49 randomly selected up- and down-regulated genes of each clone set. This literature search found a strong enrichment of genes whose products are known to confer drug resistance within the upregulated genes thus validating our strategy (Supplementary FIG. 1).

Inspection of the gene lists did not reveal an expression change in genes that are involved in Gem transport and activation and whose alteration can lead to Gem resistance (nucleoside transporters CNT1, ENT1, ENT2; deoxycytidine kinase, thymidine kinase 2, deoxycytidine deaminase and ribonucleotide reductase).

We reasoned that genes which are universally upregulated across all clone sets play dominant, central roles in the resistance mechanism. However, we did not detect single genes that were upregulated in the resistant clones of all clone sets as is often the case in cancer where the great heterogeneity of the disease prevents finding expression changes or mutations that pertain to all cancer samples. For this reason, we investigated which gene groups with a common biological function were differentially regulated in the resistant and sensitive clones. We identified differential expression in gene groups controlling DNA replication, cell death or protein synthesis (FIG. 2A). Thus, our analysis identified two known processes whose alterations are at the heart of the Gem mechanism—DNA replication and cell death. The observation that protein synthesis controlling genes are differentially expressed in Gem resistant cells is novel. We hypothesized therefore that protein synthesis regulation might be a new important element in Gem-induced cell death.

The methodology that we used for identifying differentially expressed gene groups (Goeman et al., 2004) did not establish whether the respective gene groups were up- or downregulated in the resistant clones. In order to address this question, we determined which gene groups were enriched among the upregulated genes of each clone set. This way, we found enrichment of protein synthesis controlling gene groups among the upregulated genes of all clone sets (FIG. 2B, Supplementary FIG. 2). Using quantitative real-time PCR, we confirmed the upregulation of protein synthesis controlling genes across the three clone sets in the majority of the tested genes despite the low change in expression (FIG. 2C). Thus, collectively, the microarray data strongly suggested that regulation of overall protein synthesis plays an important role in cell death induced by Gem. Since regulation of protein synthesis is so far not known to play a role in Gem- or DNA damage-induced cell death, we decided to further investigate its function in the response to Gem.

Protein synthesis in the presence of Gem is higher in the resistant clones—We first used metabolic labelling to measure the protein synthesis rates directly. Upon treatment of the 3 parental cell lines with Gem, protein synthesis was reduced. This decrease of protein synthesis was caused by inhibition of protein translation rather than enhanced protein degradation because a pulse-chase labelling revealed the same degradation rates in Gem treated and untreated cells (FIG. 3A). Reduction of protein synthesis occurred before onset of apoptosis since inhibition of apoptosis by the pan-caspase inhibitor z-VAD-fmk or Bcl-2 overexpression did not alter the protein synthesis rates with Gem (FIG. 3B). Finally, we measured the protein synthesis rates in two resistant and sensitive clones of each clone set, i.e. in 12 cell lines. We did not detect any difference in protein synthesis rates between the sensitive and resistant clones at the basal level without Gem (data not shown). However, in each clone set, Gem treatment decreased protein synthesis significantly less in the resistant clones compared to the sensitive clones. When treated with Gem, the resistant clones of HT-1197 even exhibited an increase of protein synthesis over the basal level without Gem (FIG. 3C). Hence, as inferred from the microarray data, the protein synthesis rates with Gem were indeed higher in the resistant clones indicating that the increase of protein synthesis upon Gem treatment could be a cause of resistance.

Overexpression of the translation factor EIF4E3 increases protein synthesis and inhibits cell death upon DNA damage—In order to establish whether enhanced protein synthesis is the cause or just a consequence of resistance to Gem, we attempted to simulate the enhanced protein synthesis in the clones by overexpressing eukaryotic translation initiation factor 4E family member 3 (EIF4E3) which is more strongly expressed in the resistant clones derived from HT-1197 (FIG. 2C). Although this gene is a member of the EIF4E family of proteins, it has only 21% homology with EIF4E at the amino acid level. Polyclonal EIF4E3-overexpressing T-24 showed about twice the expression level of empty vector transfected cells as judged by quantitative real-time PCR. We treated these cell lines with Gem and, in order to show a wider significance of our findings, with 3 more DNA-damaging drugs of diverse structure and mechanism of action. We used mitomycin C which alkylates DNA, etoposide which inhibits topoisomerase II and cisplatin which forms DNA adducts. Cell death of Gem, mitomycin C and etoposide, but not cisplatin was inhibited by overexpression of EIF4E3 (FIG. 4A). This inhibition of cell death, at lower concentrations, was accompanied by an increase in protein synthesis over the basal level without the drug (FIG. 4B) thus resembling the data of the HT-1197 resistant clones (FIG. 3C). Hence, DNA damage-induced increase of protein synthesis caused by overexpression of a translation factor indeed inhibits cell death. Not all DNA damage is affected in the same way by overexpression of EIF4E3. It appears that this translation factor is preferentially downstream of S-phase specific DNA damage that leads to inhibition of DNA synthesis as is the case for Gem and etoposide.

Inhibition of protein synthesis during Gem treatment reverses Gem resistance—As a next step, we set out to apply our findings for overcoming Gem resistance in the clones. Since increase of protein synthesis inhibits cell death upon DNA damage, it should be possible to improve the effect of DNA-damaging drugs by enhancing the downregulation of protein synthesis. For this reason, we treated 2 sensitive and 2 resistant clones of each clone set concomitantly with Gem and three protein synthesis inhibitors that act differently on the protein synthesis machinery. While the aminoglycoside G418 inhibits peptide chain translocation and causes codon misreading (Howard et al., 1996; Vicens & Westhof, 2003), cycloheximide inhibits peptide chain initiation (Obrig et al., 1971; Stocklein & Piepersberg, 1980) and the immunosuppressant rapamycin inhibits translation initiation via targeting the protein kinase mammalian target of rapamycin (Hay & Sonenberg, 2004). Indeed, treatment with G418 was able to overcome resistance to Gem since the combination of Gem and G418 yielded more cell death in the resistant clones than either treatment alone. In contrast, treatment with cycloheximide and rapamycin inhibited Gem-induced cell death (FIG. 5A). However, the inhibition of cell death was more pronounced in the sensitive clones, leading to a relative augmentation of cell death in the resistant clones with respect to the sensitive clones. Thus, qualitatively, addition of all three protein synthesis inhibitors led to an increase of Gem-induced cell death in the resistant clones with respect to the sensitive clones showing that protein synthesis upregulation contributes to Gem resistance. These results may be explained by the effect of the inhibitors on Gem-regulated protein synthesis. As shown in FIG. 5B, only G418 was able to further downregulate protein synthesis in the presence of Gem whereas cycloheximide treatment increased protein synthesis compared to Gem alone and rapamycin did not alter the downregulation of protein synthesis.

We also wanted to know whether the concurrent treatment with Gem and G418 could inhibit the selection of Gem-resistant clones, a process that resembles tumour recurrence in vivo. For this purpose, we repeated the selection of the parental cell lines with Gem and added G418 in a long term clonogenic survival assay. While G418 alone, at the concentrations used, did not influence cell survival, the combination of G418 with Gem completely prevented the survival of resistant clones (FIG. 5C).

G418 enhances Gem-induced cell death or growth inhibition in various cancer cell lines—Since enhancing Gem action would benefit therapy, we tested whether supplementing Gem with G418 could augment the effect of Gem also in other cancer cell lines. To this end, we added G418 at fixed concentrations and determined the EC50 value for Gem-induced cell death. In order to more clearly detect an effect of G418 on Gem-mediated growth inhibition, we used G418 concentrations that had no or little effect on their own. In each case, addition of G418 decreased the EC50 value; in some cell lines dramatically up to 30 fold. The cell dines originated from diverse types of cancer namely bladder cancer (T-24, HT-1197, and HT-1376), breast cancer (MCF-7), lung cancer (A-549) and pancreatic cancer (AsPC-1) showing the wide applicability of this drug combination and opening a possibility for improving Gem-based therapy (FIG. 6).

G418 enhances Ara-C induced cell death—In order to test whether G418 also enhances cell by other chemotherapeutic agents, we performed a dose response study using the nucleotide analog Cytarabine (Ara-C) and G418. As can be seen in FIG. 7, G418 also greatly enhanced Ara-C-mediated cell death.

### TITLES AND LEGENDS TO FIGURES

FIG. 1. The selected clones differ in Gem sensitivity but not cell proliferation. T-24 and the clones derived from T-24 were incubated with 30 nM Gem for 2 days and cell death levels were measured by propidium iodide dye exclusion using flow cytometry (A) or the number of cells without Gem was counted (B). Error bars depict Standard Error of the Mean (S.E.M.) (C) Summary of the cell death values or (D) cell proliferation values of the 3 parental cell lines, the 13 resistant and 11 sensitive clones. Lines in the boxes depict the median, boxes depict upper and lower quartile and whiskers depict maximum and minimum values. All values are from three independent experiments in duplicates.

FIG. 2. Protein synthesis controlling genes are upregulated in the resistant clones (A) Gene groups of common biological function that are differentially expressed in the sensitive and resistant clones. (B) A second analysis identified enrichment of genes with gene ontology term “protein biosynthesis” (T-24 and HT-1376 clone sets) or “translation initiation” (HT-1197 clone set) among the genes that are upregulated in the resistant clones. Depicted is the fraction among all genes on the DNA microarray that belong to these groups and the fraction among the upregulated genes only. (C) Quantitative real-time PCR of three select protein synthesis controlling genes of each clone set that were suggested to be upregulated by microarray data. Expression was normalized to expression of “house keeping genes” as described in Materials and Methods. Error bars depict S.E.M. *P<0.05, ***p<0.001.

FIG. 3. Protein synthesis in the presence of Gem is higher in the resistant clones. (A) The parental cell lines were incubated with Gemcitabine for a time that did not induce apparent cell death (T-24: 30 nM Gem, 1 day; HT-1376: 80 nM, 3 days; HT-1197: 120 nM, 3 days). Medium was removed and replaced for 1.5 h by medium containing 30 μCi/ml ³⁵S-methionine/cysteine (pulse). Medium was changed for normal growth medium containing 50 μg/ml Cycloheximide and cells grown for an additional 1.5 h (chase). The reduction in radioactively labelled proteins due to protein degradation during the chase was significant as indicated. (B) The three parental cell lines and HT-1197 and T-24 stably expressing Bcl-2 were incubated with Gem as in (A) in the presence of 40 μM (T-24, HT-1376) or 80 μM z-VAD-fmk (HT-1197) or vehicle (DMSO). Subsequently, the cells were labelled as before. (C) Two sensitive and two resistant clones of each clone set were incubated and labelled as in (A). All values were significantly different (at P<0.05) from the control values without Gem. Results are from two independent experiments in duplicate. Error bars depict S.E.M. **P<0.01.

FIG. 4. Overexpression of EIF4E3 accelerates protein synthesis and inhibits cell death upon DNA damage. (A) Polyclonal T-24 cell lines were incubated with Gem, Mitomycin C, etoposide or cisplatin for 2 days. Vehicle (DMSO) was added as a control for etoposide. Cell death was measured by propidium iodide dye exclusion using flow cytometry. (B) Polyclonal T-24 cell lines were incubated with Gem, Mitomycin C, etoposide or cisplatin for 1 day. Subsequently, the cells were metabolically labelled with 30 μCi/ml ³⁵S-methionine/cysteine. Results are from two independent experiments in duplicates. P-values were calculated by paired (A) or unpaired (B) Student's t-test. Error bars represent S.E.M. *P<0.05.

FIG. 5. Protein synthesis inhibitors diminish Gem resistance. (A) Two sensitive and two resistant clones of each clone set were incubated with Gem (T-24: 30 nM, 2 days; HT-1376: 80 nM, 4 days; HT-1197: 120 nM, 5 days). Additionally, 250 μg/ml G418 (T-24 and HT-1376 clone sets) or 70 μg/ml G418 (HT-1197 clones set), 500 ng/ml cycloheximide (CHX) or 10 nM rapamycin or DMSO as a control were added. Cell death was measured by propidium iodide dye exclusion using flow cytometry. (B) The parental cell lines were incubated with Gem (T-24: 30 nM Gem, 1 day; HT-1376: 80 nM, 3 days; HT-1197: 120 nM, 3 days) in the absence or presence of the indicated protein synthesis inhibitors (250 μg/ml G418 for T-24, 80 μg/ml for HT-1376, 70 μg/ml for HT-1197; 500 ng/ml CHX for all cell lines; 10 nM rapamycin for all cell lines). Subsequently, cells were labeled with 30 μCi/ml ³⁵S-methionine/cysteine. (C) The parental cell lines were incubated with Gem like for the selection of the Gem-resistant clones and additionally G418 was added at 250 μg/ml (T-24), 80 μg/ml G418 (HT-1376) or 70 μg/ml G418 (HT-1197). Afterward, the remaining cells on the culture plate were stained with crystal violet. A representative assay is shown. All other data are from two independent experiments in duplicate. Error bars represent S.E.M. **P-<0.01, ***p<0.001.

FIG. 6. G418 enhances Gem action in diverse cancer cell lines. (A) The cell lines were each incubated with 5 concentrations of Gem for 2 days (T-24, A-549, AsPC-1), 4 days (MCF-7, HT-1376) or 5 days (HT-1197) in the presence or absence of G418. Cell death was measured by propidium iodide dye exclusion except for MCF-7 and AsPC-1 where cell number was measured by MTT-assay. (B) Depiction of the G418 concentrations used and the basal cell death or growth inhibition with G418 only. For determination of Gem-induced cell death or growth inhibition, background values without Gem or with G418 only were subtracted. Values are from two independent experiments in duplicate. *P<0.05.

FIG. 7. G418 enhances Ara-C action. T-24 cells were incubated with 5 concentrations of Ara-C for 2 days in the presence or absence of 0.25 mg/ml G418. Cell death was measured by propidium iodide dye exclusion. For determination of Ara-C-induced cell death, background values with G418 only were subtracted. There was no increase in cell death with G418 only. Values are from two independent experiments in duplicate.

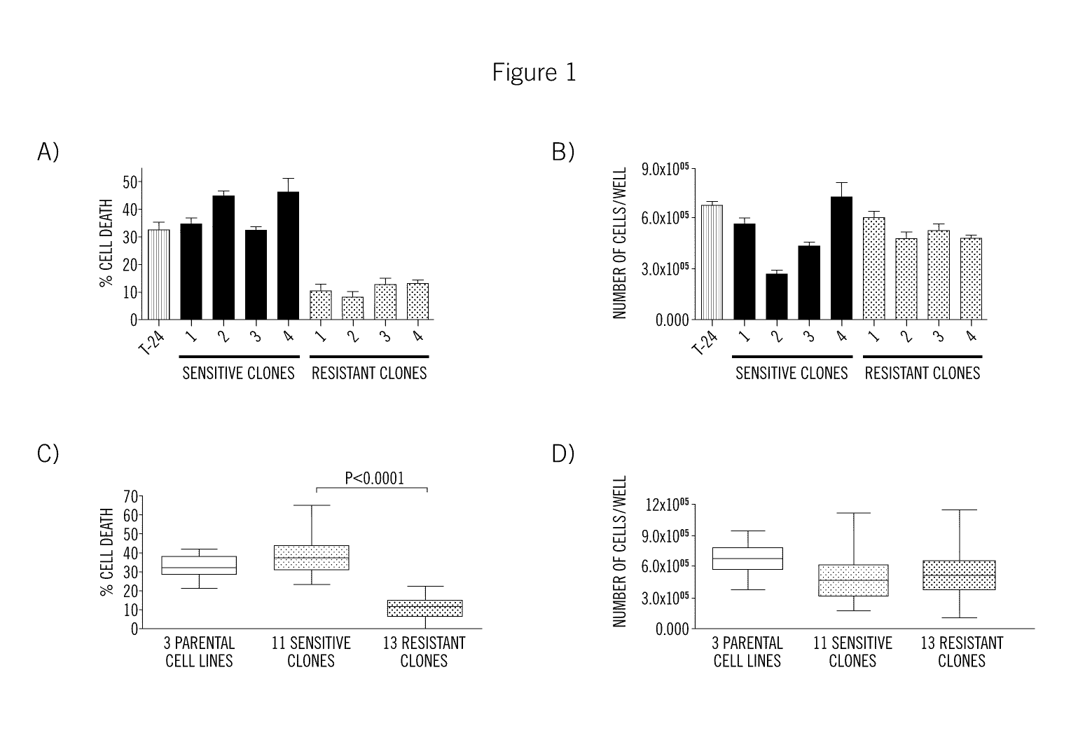

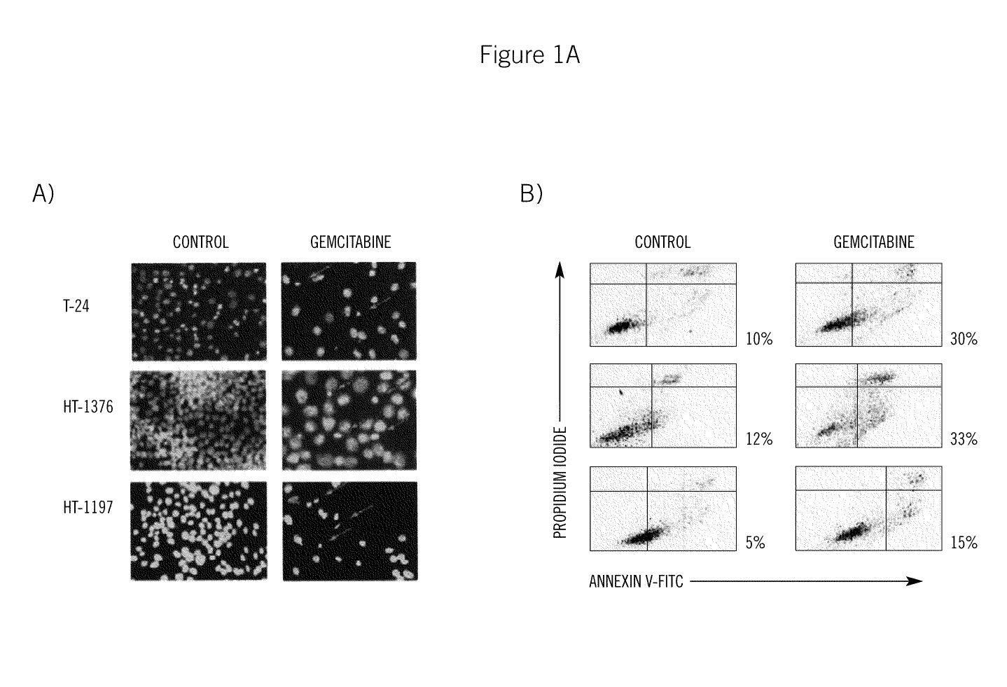

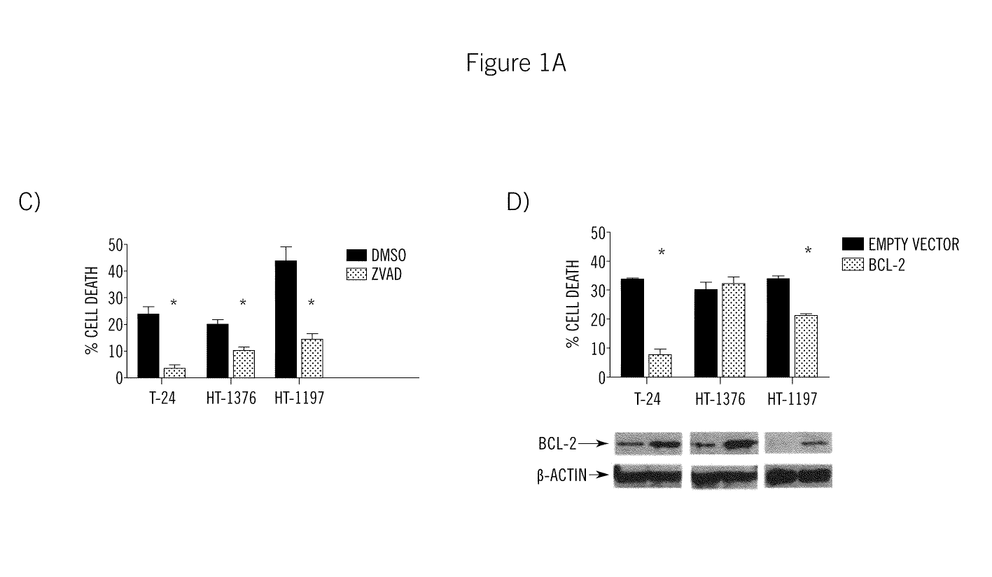

### DISCUSSION

How DNA damage leads to cell death is best known in normal cells, where the tumour suppressor p53 is functional and activated upon DNA damage, thus stimulating the transcription of pro-apoptotic genes. In cancer cells however, the p53 pathway is often deactivated by mutations in p53 or interacting proteins (Abraham et al., 2007; Vogelstein & Kinzler, 2004). Yet, a mainstay of cancer treatment—chemotherapy—is based in large part on the ability of DNA-damaging drugs to kill tumour cells. In order to improve therapy, it is therefore imperative to learn the mechanisms of DNA damage induced cell death in these cells. In our study, we identify the regulation of overall protein synthesis as a novel element of DNA damage induced apoptosis in cancer cells.

Since the inhibition of protein synthesis by DNA-damaging drugs is concentration-dependent, the weaker inhibition of protein synthesis in the Gem-resistant clones could reflect reduced sensitivity towards Gem rather than the basis of resistance. In this scenario, shut down of protein synthesis could be a means of saving energy in cells that are destined to die in any case. However, the results in the HT-1197 resistant clones contradict this interpretation because here Gem treatment even increases protein synthesis over the basal level without Gem. These results were recapitulated with Gem and 2 more DNA-damaging drugs in cells that ectopically overexpress the translation factor EIF4E3 showing that translation factors can actually cause inhibition of DNA damage-induced cell death and strongly suggesting that the inhibition proceeds via increasing protein synthesis upon DNA damage. This is the first time that increased global protein translation is reported upon DNA damage. We believe that the most straightforward interpretation of these results is analogous to events in stress signaling. When cells are exposed to stress like nutrient starvation, unfolded proteins, oxidative stress or hypoxia they react by reducing their global protein synthesis rate in order to save energy while at the same time increasing the translation of select proteins that ensure cell survival under these conditions (Holcik & Sonenberg, 2005). Our data indicate that genotoxic stress leads to similar processes, i.e. downregulation of the majority of proteins upon DNA damage and a concomitant EIF4E3-mediated upregulation of certain proteins that help survival, for instance by repairing DNA. Using metabolic labeling, the upregulation of these proteins may only be observed at lower drug concentrations because at higher concentrations, the reduction of global protein synthesis masks the increased translation of these proteins.

Protein synthesis in stress signaling is reduced via phosphorylation of eukaryotic translation initiation factor 2α (eIF2α). However, neither eIF2α nor eukaryotic initiation factor 4E binding protein 1 nor ribosomal protein S6 changed phosphorylation state upon Gem treatment. Hence, the question as to how protein synthesis is regulated upon DNA damage is still open.

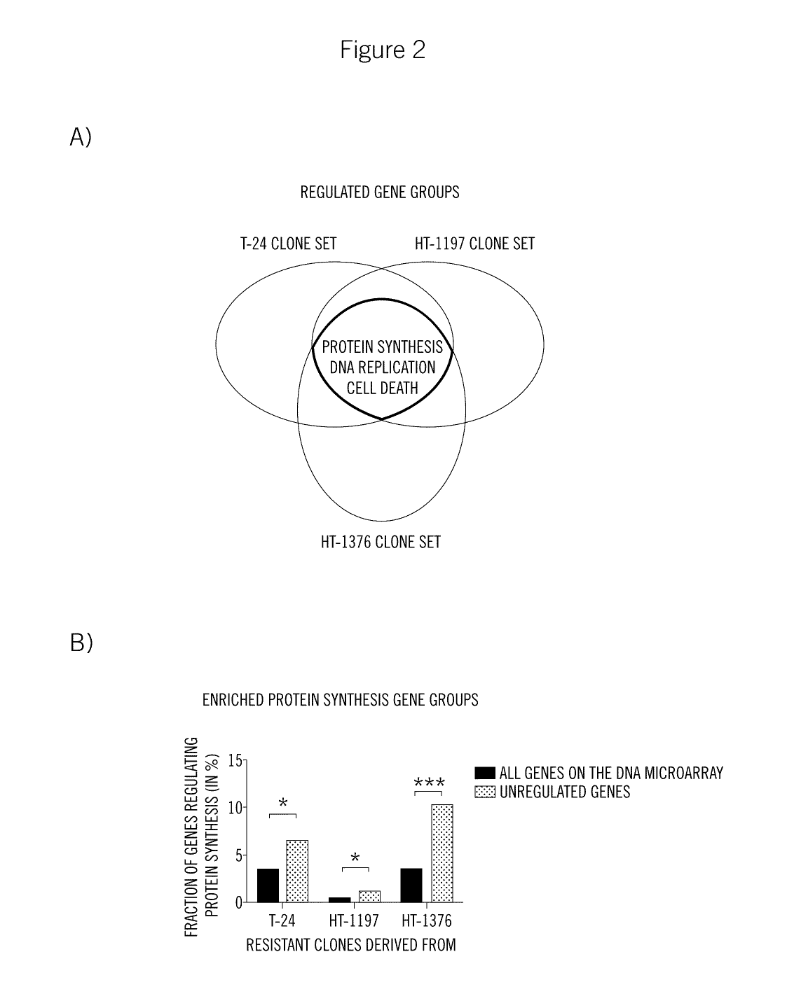

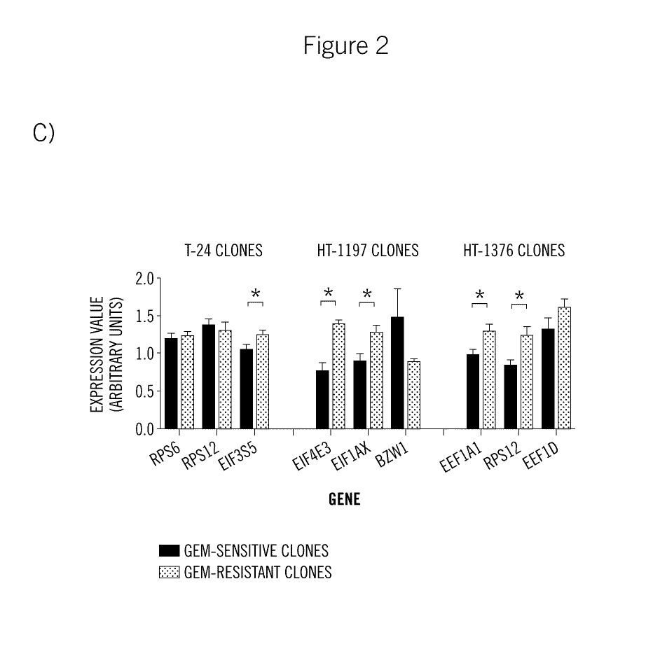

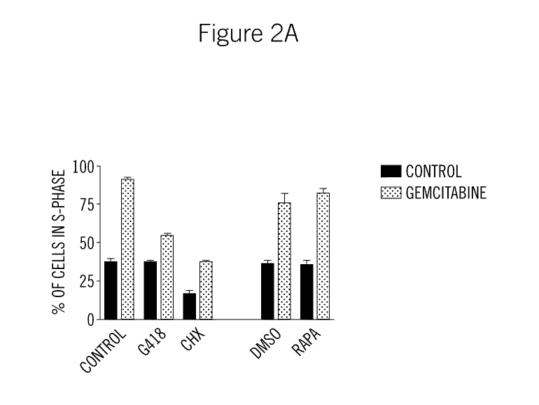

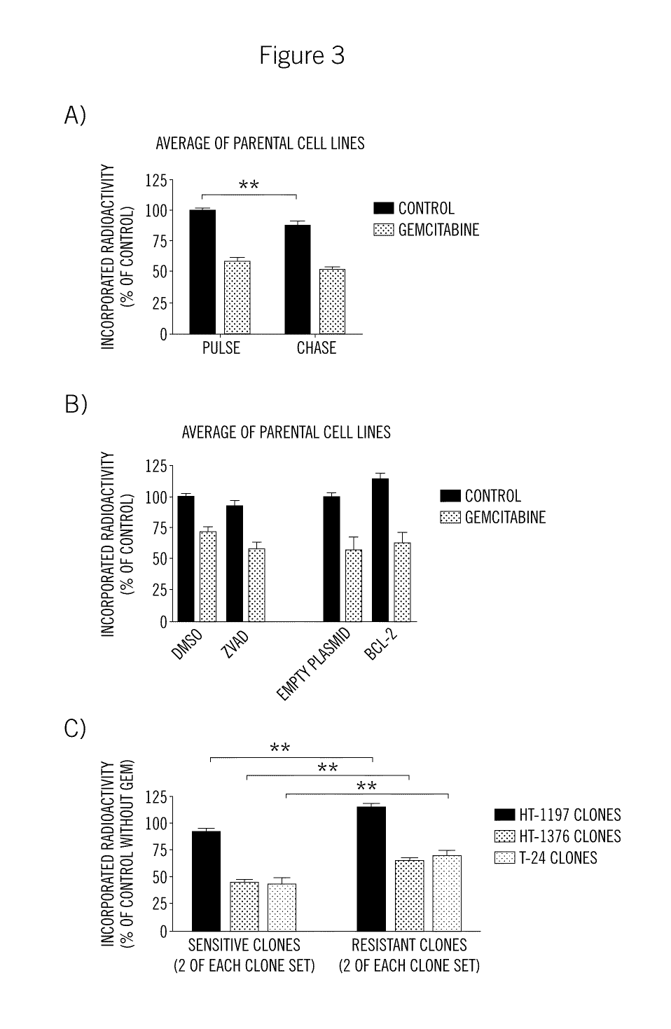

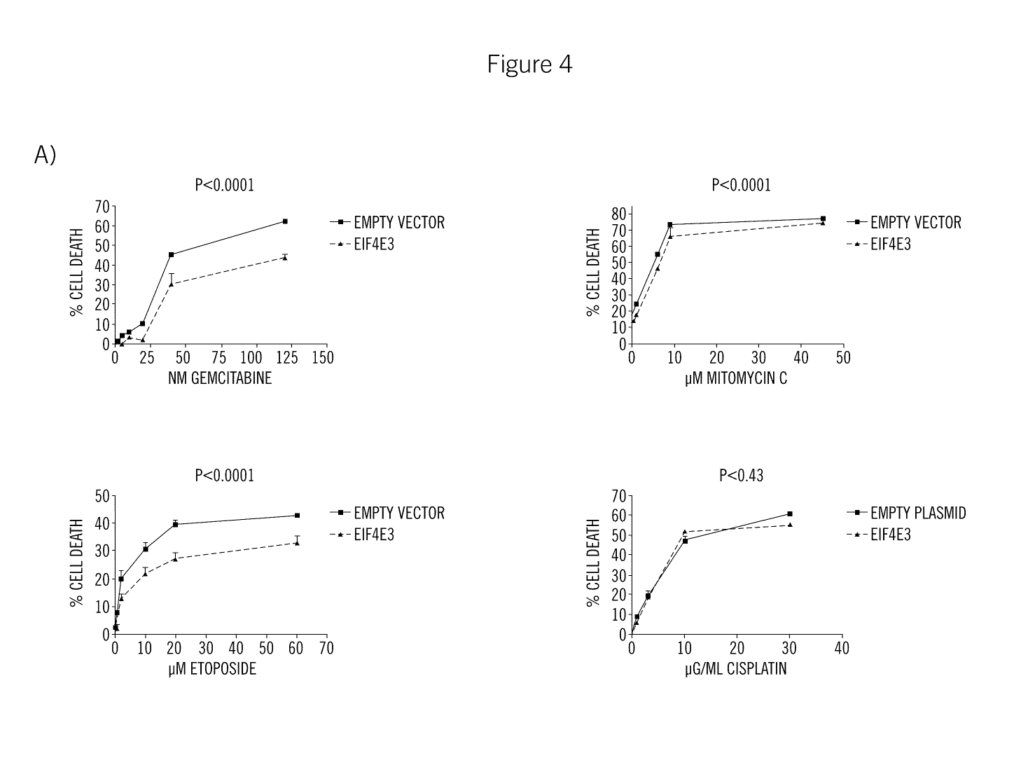

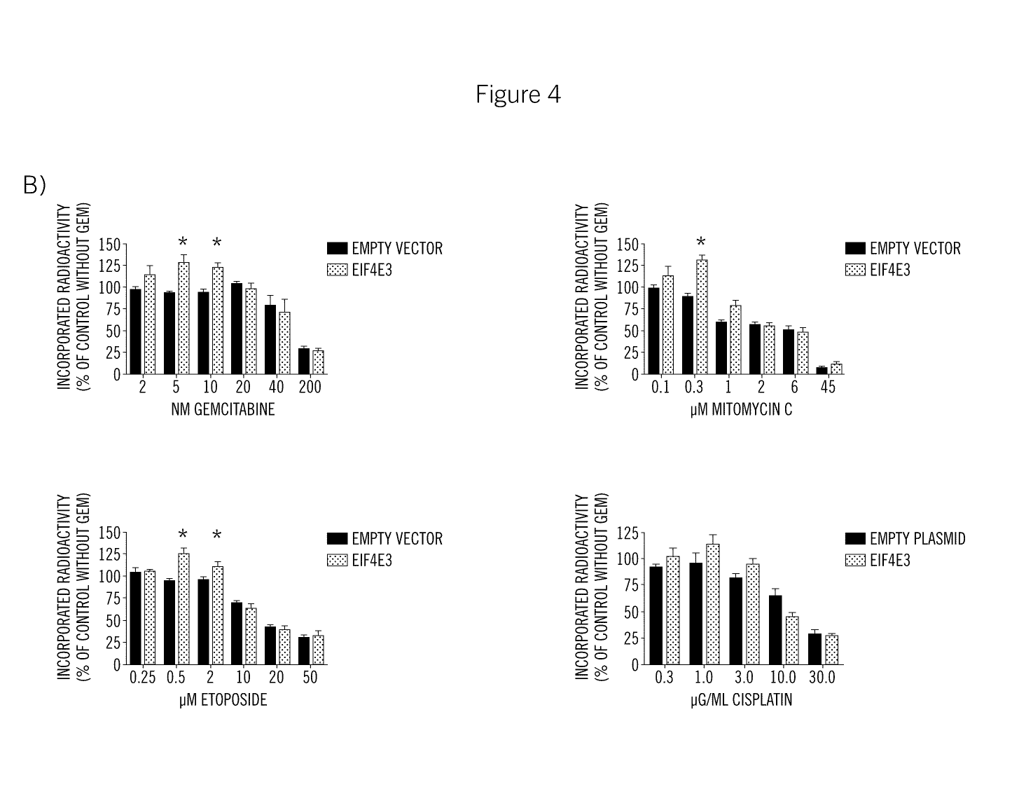

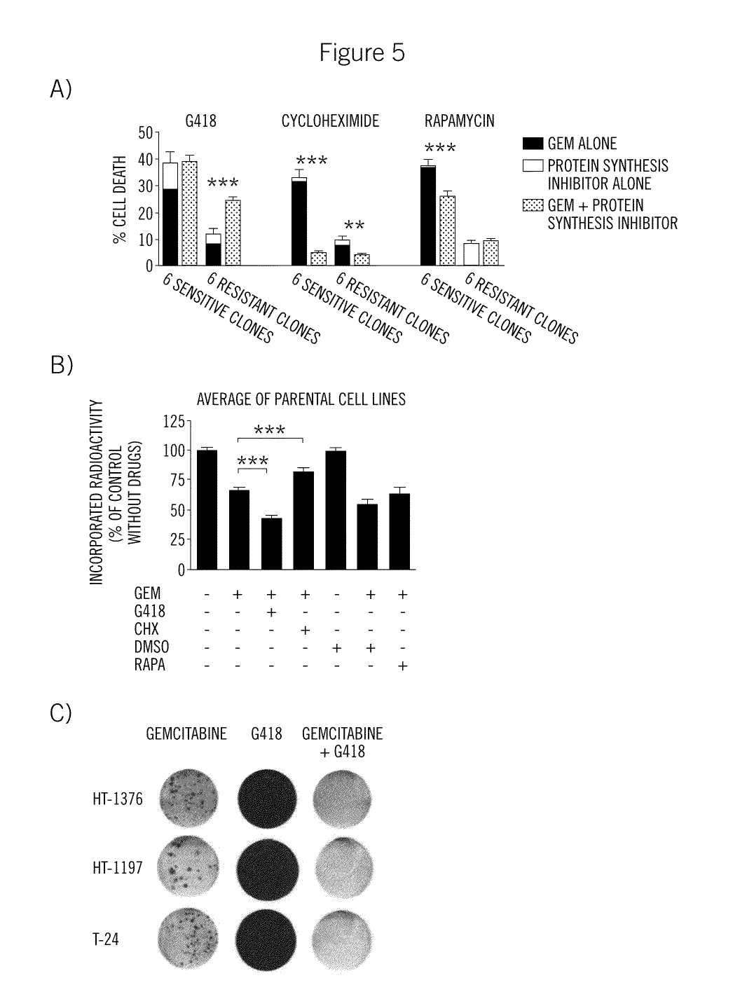

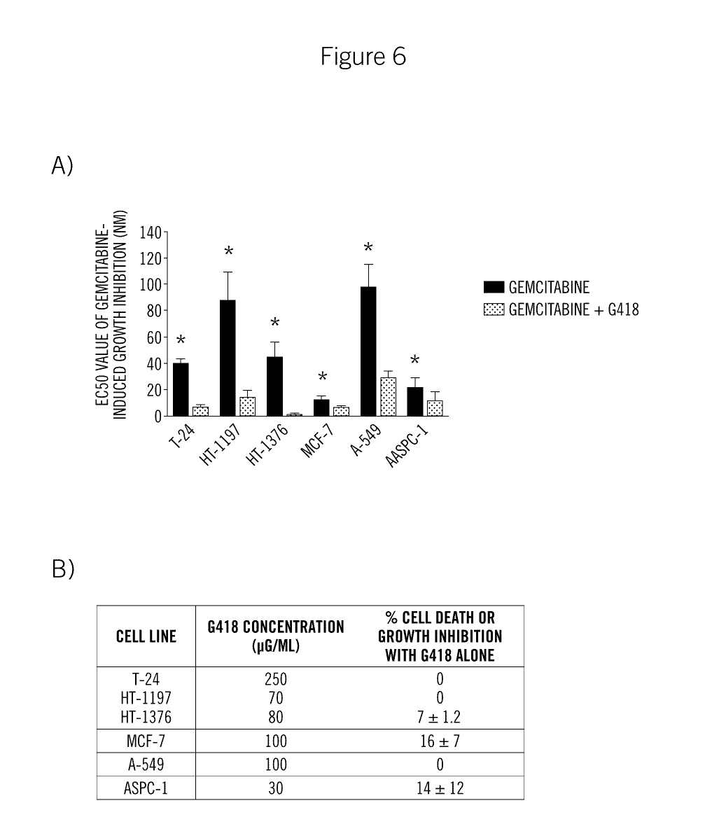

Interactions between two inhibitors, especially broad-spectrum inhibitors like DNA damaging agents and protein synthesis inhibitors are hard to predict. Consequently, working drug combinations are commonly found by “trial and error” approaches in vitro or in vivo using combinations of (usually already clinically used) drugs with little beforehand guidance about their interaction. Despite these difficulties, we could show a common outcome when combining protein synthesis inhibitors with Gem. When using this combination, the difference in Gem-induced cell death between the resistant and sensitive clones became smaller with each protein synthesis inhibitor used showing that increased protein synthesis is at least partially responsible for the resistant phenotype in the clones. The decreased Gem-induced cell death, particularly when using cycloheximide might be attributed to “unwanted” downregulation of proteins that are necessary for cell death induction by Gem. In line with this explanation is a reduced fraction of cells in S-phase upon treatment with cycloheximide alone which interferes with incorporation of Gem into DNA (Supplementary FIG. 2) and an even increased protein synthesis rate when compared to Gem alone. Similarly, the trade-off between wanted and un-wanted effects may have prevented downregulation of protein synthesis and increase of cell death when we used G418 with other DNA damage-inducing drugs but Gem (data not shown).

Despite the well-known side effects of aminoglycoside antibiotics like G418, a combination with Gem need not be too toxic. G418 or other as yet untested aminoglycosides may be used at low enough concentrations to allow an effect on the cancer without aminoglycoside-based side effects. Treatment of mice with G418 at concentrations comparable to the concentrations used by us has been done without detectable effects on mice (Murphy et al., 2006). Additionally, protein synthesis inhibitors like G418 may preferentially affect tumour cells because protein synthesis is upregulated in cancer.

The results presented here, illustrate the complex nature of the DNA damage response and how regulation of seemingly broad processes like protein synthesis can have very specific phenotypic effects. Beside the impact on well-known effectors of DNA damage like p53 and abl, there are underlying alterations in protein translation that influence a large number of proteins. Hence, for a complete understanding of DNA damage signaling, it will be pivotal to identify the proteins that are regulated by the changes in protein synthesis rates. These new avenues of research will lead to a better understanding of DNA damage-induced death of tumour cells, a knowledge that may ultimately lead to better cancer therapies.

### REFERENCES

Abraham, R., Pagano, F., Gomella, L. G. & Baffa, R. (2007). Front Biosci, 12, 826-38.
Abraham, R., Schafer, J., Rothe, M., Bange, J., Knyazev, P. & Ulirich, A. (2005). J Biol Chem, 280, 34123-32.
Barton-Burke, M. (1999). Cancer Nurs, 22, 176-83.
Benjamini, Y. & Hochberg, Y. (1995). J R Statist SocB, 57, 289-300.
Bergman, A. M., Eijk, P. P., Ruiz van Haperen, V. W., Smid, K., Veerman, G., Hubeek, I., van den Ijssel, P., Ylstra, B. & Peters, G. J. (2005). Cancer Res, 65, 9510-6.
Bergman, A. M., Pinedo, H. M. & Peters, G. J. (2002). Drug Resist Updat, 5, 19-33.
Bubenik, J., Baresova, M., Viklicky, V., Jakoubkova, J., Sainerova, H. & Donner, J. (1973). Int J Cancer, 11, 765-73.
Davis, R. J. (2000). Cell, 103, 239-52.
De Benedetti, A. & Graff, J. R. (2004). Oncogene, 23, 3189-99.
Goeman, J. J., van de Geer, S. A., de Kort, F. & van Houwelingen, H. C. (2004). Bioinformatics, 20, 93-9.
Harris, S. L. & Levine, A. J. (2005). Oncogene, 24, 2899-908.
Hay, N. & Sonenberg, N. (2004). Genes Dev, 18, 1926-45.
Holcik, M. & Sonenberg, N. (2005). Nat Rev Mol Cell Biol, 6, 318-27.
Howard, M., Frizzell, R. A. & Bedwell, D. M. (1996). Nat Med, 2, 467-9.
Huang, H., Regan, K. M., Lou, Z., Chen, J. & Tindall, D. J. (2006). Science, 314, 294-7.
Kang, Y., Siegel, P. M., Shu, W., Drobnjak, M., Kakonen, S. M., Cordon-Cardo, C., Guise, T. A. & Massague, J. (2003). Cancer Cell, 3, 537-49.
Kastan, M. B. & Bartek, J. (2004). Nature, 432, 316-23.
Kharbanda, S., Pandey, P., Jin, S., Inoue, S., Bharti, A., Yuan, Z. M., Weichselbaum, R., Weaver, D. & Kufe, D. (1997). Nature, 386, 732-5.
Murphy, G. J., Mostoslavsky, G., Kotton, D. N. & Mulligan, R. C. (2006). Nat Med, 12, 1093-9.
Nicoletti, I., Migliorati, G., Pagliacci, M. C., Grignani, F. & Riccardi, C. (1991). J Immunol Methods, 139, 271-9.
Obrig, T. G., Culp, W. J., McKeehan, W. L. & Hardesty, B. (1971). J Biol Chem, 246, 174-81.
Plunkett, W., Huang, P., Searcy, C. E. & Gandhi, V. (1996). Semin Oncol, 23, 3-15.
Rasheed, S., Gardner, M. B., Rongey, R. W., Nelson-Rees, W. A. & Arnstein, P. (1977). J Natl Cancer Inst, 58, 881-90.
Ruggero, D. & Pandolfi, P. P. (2003). Nat Rev Cancer, 3, 179-92.
Stocklein, W. & Piepersberg, W. (1980). Antimicrob Agents Chemother, 18, 863-7.
Stoneley, M. & Willis, A. E. (2004). Oncogene, 23, 3200-7.
Tee, A. R. & Proud, C. G. (2000). Oncogene, 19, 3021-31.
Vicens, Q. & Westhof, E. (2003). J Mol Biol, 326, 1175-88.
Vogelstein, B. & Kinzier, K. W. (2004). Nat Med, 10, 789-99.
Vousden, K. H. (2000). Cell, 103, 691-4.
Yuan, Z. M., Huang, Y., Ishiko, T., Kharbanda, S., Weichselbaum, R. & Kufe, D. (1997). Proc Natl Acad Sci USA, 94, 1437-40.
Zhang, Y., Yan, J., Chen, K., Song, Y., Lu, Z., Chen, M. & Han, C. (2004). Clin Exp Pharmacol Physiol, 31, 626-33.

1. A method of treatment of a subject affected by neoplastic diseases comprising the administration to said subject of an aminoglycoside antibiotic in combination with a nucleoside analogue.

2. A method according to claim 1 wherein the nucleoside analogue is gemcitabine.

3. A method according to claim 1 wherein the nucleoside analogue is Ara-C.

4. A method according to claim 1 wherein the aminoglycoside antibiotic is Geneticin (G418).

5. A method according to claim 1 wherein the neoplastic disease is selected from non-small cell lung cancer, pancreatic cancer, bladder cancer, breast cancer and ovarian cancer.

6. Pharmaceutical compositions comprising a nucleoside analogue and an aminoglycoside antibiotic as the active ingredients.

7. Pharmaceutical compositions according to claim 6 wherein the nucleoside analogue is gemcitabine or Ara-C and the aminoglycoside antibiotic is geneticin.

8. Pharmaceutical compositions according to claim 6 in form of kit-of-parts containing gemcitabine or Ara-C and geneticine as combined preparation for the simultaneous, separate or sequential administration.

9. Pharmaceutical compositions according to claim 7 in form of kit-of-parts containing gemcitabine or Ara-C and geneticine as combined preparation for the simultaneous, separate or sequential administration.
